(19) United States
(12) Patent Application Publication
(10) Pub. No.: US 2021/0040533 A1
(43) Pub. Date: Feb. 11, 2021

(54) METHOD FOR IDENTIFYING TARGET GENE OF TEST SUBSTANCE

(71) Applicant: DRUG GENOMICS, LTD., Chiba-shi (JP)

(72) Inventor: Hiroji CHIBANA, Chiba-shi (JP)

(73) Assignee: DRUG GENOMICS, LTD., Chiba-shi (JP)

(21) Appl. No.: 16/971,610

(22) PCT Filed: Feb. 20, 2019
(86) PCT No.: PCT/JP2019/006191

§ 371 Date: Aug. 20, 2020

(30) Foreign Application Priority Data
Feb. 21, 2018 (JP) 2018-029013

(51) Int. Cl. C12Q 1/18; C12N 15/81

(52) U.S. Cl. CPC C12Q 1/18; C12N 15/81

(57) ABSTRACT

Provided is an efficient and highly versatile method for identifying a target gene of a test substance. A method for identifying a target gene of a test substance previously confirmed to have a predetermined medicinal effect, comprising: culturing a modified cell in which at least one selected from the group consisting of a candidate target gene, a cis-element thereof and a trans-element thereof has been modified, or a cell in which expression of a candidate target gene has been suppressed by RNA interference, in the presence of the test substance; and detecting the modified cell or the cell having a decreased biological activity.

### TECHNICAL FIELD

The present invention relates to a method for identifying a target gene of a test substance previously confirmed to have a predetermined medicinal effect.

### BACKGROUND ART

With respect to a drug having an effect on a disease, it is important to check as to what kind of molecule (a target molecule such as a protein, a nucleic acid, a lipid) in intracellular or extracellular sections the drug acts on; how to change the function of the molecule; and how the change connects with a medicinal effect, as information for elucidating mechanism of drug action. In conventional drug development through compound-screening (phenotype screening) based on function in a living body as an index, which is a target molecule and how to act are unknown in the stage of screening; and a number of drugs are still acting on unknown target molecules.

In developing a more effective drug, it is necessary to identify a target molecule of the drug. Examples of a method for identifying a target molecule so far known include molecular biological methods such as analyses for changes in gene expression, protein expression and signal transduction; methods using, e.g., a transformant; and methods of identifying a target molecule interacting with a drug labeled with biotin or the like. However, these methods require a lot of time and labor due to the complicated network within a cell. For the reason, it has been desired to develop an efficient and highly versatile method for identifying a target molecule.

Among the pathogenic fungi including candida, some fungi cause serious opportunistic infection. As the number of patients susceptible to infection increases with aging of population, an increase in the number of mycosis patients is inevitable. Despite the situation, antifungal drugs that can be systemically administered are only 4 types. In addition, problems such as adverse effects and emergence of resistant strains are present. For the reason, there is an urgent need to develop a new antifungal drug. However, from the results of genome sequence analysis for various fungi including candida, it has been found that many proteins have amino acid sequence homology between a human and a fungus. It is thus concerned that the substances having antifungal activity may produce adverse effects on humans in many cases. If a target molecule is identified in the development stage of an antifungal drug, adverse effects can be avoided. In view of this, it has been desired to develop an efficient and highly versatile method for identifying a target molecule.

Up to present, as a method for determining a target of a compound, there has been proposed, for example, a method including providing a library of cells in which expression of a gene product of a gene involved in an essential cellular process has been controlled, exposing the library to the compound, and assaying cell proliferation (Patent Literature 1). However, the literature does not disclose how to control expression of a gene product in determining a target of a compound.

### CITATION LIST

### Patent Literature

Patent literature 1: JP-A-2002-511239

### SUMMARY OF INVENTION

### Technical Problem

An object of the present invention is to provide an efficient and highly versatile method for identifying a target gene of a test substance that is previously confirmed to have a predetermined medicinal effect.

### Solution to Problem

The present inventor conducted intensive studies with a view to attaining the above object. The inventor found that when a modified cell, which is obtained by modifying at least one selected from the group consisting of a candidate target gene, a cis-element thereof and a trans-element thereof, or a cell, which is obtained by suppressing expression of a candidate target gene by RNA interference, is cultured in the presence of a test substance, the test substance significantly produces its effect on the modified cell or the cell; and that a target gene of the test substance can be efficiently identified by detecting the modified cell or the cell whose biological activity decreases after culture. More specifically, the inventor found that when a modified cell, which is obtained by inserting an inducible promoter into a site upstream of a candidate target gene, is cultured in the presence of an inducer in a concentration that is 0.001 times or more and 10 times or less of IC₅₀, which is a concentration at which biological activity of the modified cell is inhibited by 50%, and a test substance in a concentration that is 0.001 times or more and 10 times or less of IC₅₀, which is a concentration at which biological activity of a cell, in which expression of a candidate target gene is not regulated, is inhibited by 50%, the inducer and the test substance produce a synergetic effect on the modified cell; and that a target gene of the test substance can be efficiently identified by detecting the modified cell whose biological activity decreases after culture. On the basis of these findings, the present invention was accomplished.

Accordingly, the present invention provides the following [1] to [11].

[1] A method for identifying a target gene of a test substance previously confirmed to have a predetermined medicinal effect, comprising: culturing a modified cell in which at least one selected from the group consisting of a candidate target gene, a cis-element thereof and a trans-element thereof has been modified, or a cell in which expression of a candidate target gene has been suppressed by RNA interference, in the presence of the test substance; and detecting the modified cell or the cell having a decreased biological activity.
[2] The method according to [1], wherein the modified cell comprises an inducible promoter inserted into a site upstream of the candidate target gene.
[3] The method according to [2], wherein the culturing of the modified cell comprises culturing the modified cell in the presence of an inducer in a concentration that is 0.001 times or more and 10 times or less of IC₅₀, which is a concentration at which biological activity of the modified cell is inhibited by 50%, and the test substance in a concentration that is 0.001 times or more and 10 times or less of IC₅₀ which is a concentration at which biological activity of a cell in which expression of the candidate target gene is not regulated is inhibited by 50%.
[4] The method according to [2], wherein the culturing of the modified cell comprises culturing the modified cell in the presence of the test substance in a concentration that is 0.001 or more and 10 times or less of IC; which is a concentration at which biological activity of a cell in which expression of the candidate target gene is not regulated is inhibited by 50% when biological activity of the modified cell decreases to 50% or less compared to the cell in which expression of the candidate target gene is not regulated.
[5] The method according to [2], wherein the culturing of the modified cell comprises culturing the modified cell in the presence of an inducer in a concentration that is not less than EC₅₀, which is a concentration at which biological activity of the modified cell decreases to 50% of a maximum, and 100 times or less of EC₅₀ and the test substance in a concentration that is 0.001 times or more and 10 times or less of IC₅₀, which is a concentration at which biological activity of a cell in which expression of the candidate target gene is not regulated is inhibited by 50%.
[6] The method according to [3], wherein the concentration of the inducer is 0.01 times or more of IC₅₀ and less than or equal to IC₅₀.
[7] The method according to [3] or [4], wherein the inducible promoter is Tet-Off (registered trademark) promoter.
[8] The method according to any one of [1] to [7], wherein the test substance has at least one medicinal effect selected from the group consisting of those of an antibacterial drug, an antifungal drug, an antineoplastic drug and an antiviral drug.
[9] The method according to any one of [1] to [8], wherein the decreased biological activity is a decreased cell proliferative activity.
[10] The method according to any one of [1] to [9], wherein the cell is an eukaryotic cell.
[11] The method according to any one of [1] to [10], wherein the cell is a fungal cell.

### Effects of Invention

According to the method of the present invention, it is possible to identify a target gene of a test substance previously confirmed to have a predetermined medicinal effect in an efficient and highly versatile manner. In the method of the present invention, a modified cell or a cell can be cultured and detected in a short time. Because of this, even if a test substance is relatively unstable, a target gene thereof can be identified. Identification of the target gene of a test substance leads to elucidation of action mechanism of the test substance and efficient development of a more effective drug, and further proceeds elucidation of action mechanism of existing drugs, contributing to, e.g., new drug development, existing drug redevelopment (drug repositioning), understanding of a mechanism of a disease of an unknown cause, and determination of causes of adverse effects.

### BRIEF DESCRIPTION OF DRAWINGS

FIG. 1 shows the Tet-Off system for use in target-gene identification in Candida glabrata.

FIG. 2 relates to identification of a target gene of an antifungal drug, fluconazole by use of tetracycline transcriptional repression strain (let strain) of Candida glabrata.

FIG. 3 relates to identification of a target gene of fluconazole by use of Tet strain of Candida glabrata.

FIG. 4 relates to identification of a target gene of fluconazole by use of Tet strain of Candida glabrata.

FIG. 5 relates to identification of a target gene of fluconazole by use of let strain of Candida glabrata.

FIG. 6 relates to identification of a target gene of an antifungal drug, terbinafine, by use of Tet strain of Candida glabrata.

### DESCRIPTION OF EMBODIMENTS

The method for identifying a target gene of a test substance of the present invention comprises: culturing a modified cell in which at least one selected from the group consisting of a candidate target gene, a cis-element thereof and a trans-element thereof has been modified, or a cell in which expression of a candidate target gene has been suppressed by RNA interference, in the presence of the test substance; and detecting the modified cell or the cell having a decreased biological activity.

The “test substance” to be used in the present invention is a substance to be administered to a living organism, preferably a mammal and more preferably a human, and is not particularly limited as long as it is a substance previously confirmed to have a medicinal effect. The test substance may be a novel substance, a known substance, a naturally occurring substance or a substance artificially synthesized by, e.g., a chemical or biological method. The test substance may be a compound, a composition or a mixture. Specifically, examples of the test substance include a nucleic acid, a carbohydrate, a lipid, a protein, a peptide, an organic compound, an inorganic compound, a microorganism, an animal/plant derived component (for example, a dried component, an extracted component, a fermented component, and a culture supernatant) and compositions containing these.

The medicinal effect of a test substance, in other words, the efficacy as a drug, is not particularly limited. Examples of the medicinal effect include those of an analgetic drug, an anesthetic drug, an anti-addiction drug/substance-abuse therapeutic drug, an antibacterial drug, an anticonvulsant drug, an anti-dementia drug, an antidepressant drug, an antiemetic drug, an antifungal drug, an antigout drug, anti-inflammatory drug, an anti-migraine drug, a myasthenic medicine, an anti-mycobacterial drug, an antineoplastic drug, an antiparasitic drug, an anti-Parkinson's disease drug, an antipsychotic drug, an antispasmodic drug, an antiviral drug, an anti-anxiety drug, a bipolar disorder therapeutic drug, a blood sugar regulator, a blood product/blood regulator/plasma volume expander, a cardiovascular drug, an agent affecting the central nervous system, a dental medicine for oral cavity, a dermatological drug, a gastrointestinal drug, a drug for the genitourinary system, a hormone activator/drug for hormonal replacement/hormone regulator, a hormone suppressor, an immunologic drug, an inflammatory bowel disease therapeutic drug, a metabolic bone disease therapeutic drug, an ophthalmic drug, an otological preparation, a drug for the respiratory system, a skeletal muscle relaxant and a sleep disorder therapeutic drug. Of the drugs, an antibacterial drug, an antifungal drug, an antineoplastic drug and an antiviral drug are preferable and an antifungal drug is further preferable.

In the present invention, the “target gene” refers to a gene on which a test substance acts, producing a medicinal effect. Examples of the target gene herein include, in addition to a gene encoding a target molecule on which a test substance directly acts, a target-related gene such as a gene encoding a molecule associating with a target molecule, a gene encoding a molecule activating or suppressing a target molecule, and a gene encoding a molecule required for constructing a molecule activating or suppressing a target molecule. A candidate target gene is not limited to the gene encoded by the genome of a cell from which the modified cell to be used in the present invention is derived or the cell to be used in the present invention. A heterogeneous or homogeneous knock-in gene is also included. Of them, a gene influencing biological activity of a cell is preferable, and a gene essential for cell growth is further preferable because a target gene can be easily identified based on cell proliferation activity as an index. The gene essential for cell growth refers to a gene required for growth/proliferation of a cell, in other words, refers to a gene causing growth retardation or inviability of the cell if the gene is destroyed.

In the present invention, the “inducible promoter” refers to a promotor that can regulate expression of a gene operably linked thereto, based on the presence or absence of an inducer externally added or stimulation externally applied. The expression “operably linked” used herein means that a regulatory region (e.g., promoter) and a coding region of a gene are arranged in an appropriate positional relationship such that expression of the gene is regulated by the function of the regulatory region. The regulation of gene expression encompasses induction, augmentation, suppression, inhibition, and the like of gene transcription.

The types of an inducible promoter and an inducer or an inductive stimulus can be appropriately selected in consideration of, e.g., the type of cell to which the promoter is to be introduced, the effect on cell proliferation, toxicity to a cell and convenience for operation. Examples of the inducible promoter include, but are not limited to, a lactose inducible promoter (e.g., lac promoter, lacUV5 promoter, tac promoter, trc promoter, Pspac promoter), a galactose inducible promoter (e.g., gall promoter, gal4 promoter, gal10 promoter, mel1 promoter), a xylose inducible promoter (e.g., xylA promoter, xylB promote), an arabinose inducible promoter (e.g., araBAD promoter), a rhamnose inducible promoter (e.g., rhaBAD promoter), a tetracycline inducible promoter (e.g., tet promoter), a hormone inducible promoter (e.g., MMTV promoter), a metal-inducible promoter (e.g., metallothionein promoter), an alcohol inducible promoter (e.g., alcA promoter), a temperature inducible promoter (e.g., λpL promoter, λpR promoter), a heat shock inducible promoter (e.g., hsp70 promoter) and a light-inducible promoter (rbcS promoter). Of the inducible promoters, a promoter that can regulate (for example, induce or inhibit) expression of a gene under control by addition of an inducer, is preferable in view of convenience of operation, and a promoter that induces expression of a gene under control in the absence of an inducer and suppresses expression of the gene under control in the presence of the inducer in a dose dependent manner, is more preferable in view of accuracy of target gene identification. More specifically, a tetracycline inducible promoter, that is, a promotor in the Tet-Off (registered trademark) system, which induces expression of a gene under control in the absence of an inducer, i.e., tetracycline or a derivative thereof, and suppresses the expression in the presence thereof, is preferable. Examples of tetracycline or a derivative thereof include tetracycline, doxycycline, chlortetracycline and oxytetracycline. In view of strength of induction activity, doxycycline is preferable.

The “modified cell” to be used in the present invention is not limited as long as it is a modified cell in which at least one selected from the group consisting of a candidate target gene, a cis-element thereof and a trans-element thereof has been modified; and preferably a modified cell in which the expression level of a candidate target gene has been increased or decreased by modification and the sensitivity to a test substance has been enhanced compared to a cell in which expression of the candidate target gene is not regulated. The phrase “sensitivity of a modified cell to a test substance has been enhanced compared to a cell in which expression of a candidate target gene is not regulated” means that, for example, when a modified cell and a cell in which expression of a candidate target gene is not regulated are separately cultured in the presence of a test substance of the same concentration and biological activity is measured, the biological activity of the modified cell is low compared to that of the cell in which expression of a candidate target gene is not regulated.

Herein, the cis-element of a candidate target gene refers to a region positioned (at a cis position) on the same molecule as the candidate target gene, more specifically positioned in a 5′ untranslated region, a 3′ untranslated region or an intron and influencing the transcriptional activity of the gene. Examples of the cis-element include, but are not limited to, an operator, a promoter, a TATA box, a CAT box and an enhancer. The trans-element of a candidate target gene refers to another gene or a gene expression product (for example, transcription factor) influencing the expression of the candidate target gene and regulating transcription of the gene through the base sequence of a cis-element.

The “modification” of at least one selected from the group consisting of a candidate target gene, a cis-element thereof and a trans-element thereof refers to changing the DNA sequence of at least one selected from the group consisting of a candidate target gene, a cis-element thereof and a trans-element thereof by a method known to those skilled in the art, such as gene recombination, genome editing, self-cloning and point mutation. For example, a case where a base(s) (e.g., about 1 to 20, preferably 1 to 10, more preferably 1 to 5 bases) is substituted, deleted, added and/or inserted in the corresponding region, may be mentioned.

The “cell in which expression of a candidate target gene is not regulated” is not limited as long as it is a cell where the expression of a candidate target gene is not enhanced or suppressed compared to that of a cell of a wild strain. Examples thereof include a cell of a wild strain, a host cell of a modified cell, a cell obtained by reintroducing a candidate target gene into a modified cell and a cell obtained by introducing a transformation marker alone into a wild type cell. Of them, a wild-strain cell is preferable in view of availability.

As the “modified cell”, a cell obtained by introducing an inducible promoter into a site upstream of the candidate target gene, is preferable. More specifically, a regulatory region containing an inducible promoter is preferably inserted in place of the original promoter of a candidate target gene such that the promoter and the candidate target gene are operably linked. The regulatory region may include, if necessary, regulatory sequences such as an operator, an enhancer, a ribosome binding site and a terminator; various types of sequences known to those skilled in the art such as a restriction enzyme cleavage site, a transformation marker, a signal sequence and a leader sequence. Of these, a transformation marker is preferably included in order to facilitate selection of a modified cell through screening. Examples of the transformation marker include, but are not limited to, a drug resistance marker gene such as a chloramphenicol resistance gene, an ampicillin resistance gene, a kanamycin resistance gene, a tetracycline resistance gene, a spectinomycin resistance gene, a streptomycin resistance gene, a neomycin resistance gene and a hygromycin resistance gene; and an auxotrophy marker gene such as a leucine synthase gene, a histidine synthase gene, a tryptophan synthase gene, a lysine synthase gene, a methionine synthase gene, an adenine synthase gene and an uracil synthase gene. In addition, in order to avoid unintended expression influenced by a sequence upstream of an inducible promoter, a terminator sequence may be arranged upstream of the inducible promoter. These various types of sequences can be appropriately selected depending on the conditions such as the type of cell to be introduced, the type of inducible promoter and the culture medium, and put in use by those skilled in the art.

A means for inserting an inducible promoter into a site upstream of a candidate target gene in a cell is not particularly limit, and a known method can be used. As an example of the known method, a method using homologous recombination will be described below; however, the method for preparing a modified cell in the present invention is not limited to this.

First, an expression vector containing an inducible promoter is prepared. To an appropriate expression vector, an inducible promoter and, if necessary, a regulatory region including, e.g., a regulatory sequence for the promoter and a transformation marker, are cloned by a genetic engineering method known to artisans. In the regulatory region, the regulatory sequence is arranged such that the promoter can function and the transformation marker is preferably arranged upstream on the 5′ side of the promoter so as not to interfere with the function of the promoter. Since various types of expression vectors having inducible promoters cloned therein are commercially available, a commercially available expression vector may be used.

Subsequently, a DNA cassette is prepared, which contains a region homologous to genomic DNA of the 5′ flanking region of a candidate target gene (homologous region A), a regulatory region containing the inducible promoter as mentioned above, and the like, and a region homologous to genomic DNA of the 5′ terminal region of ORF of a candidate target gene (homologous region B). Herein, the 5′ flanking region of a candidate target gene refers to a flanking region upstream of the initiation codon of the candidate target gene. As homologous region A, it is preferred to select a region in which original promoter activity for the candidate target gene disappears after homologous recombination but an influence on expression of other genes is minimized. The 5′ terminal region of ORF of a candidate target gene refers to a region downstream from the initiation codon of the candidate target gene.

PCR is carried out by using an expression vector having the regulatory region cloned therein as a template and a primer pair for amplifying the regulatory region, more specifically, a pair of a primer having a DNA sequence homologous to genomic DNA of the 5′ flanking region of a candidate target gene at its 5′ terminal, and a primer having a DNA sequence homologous to genomic DNA of the 5′ terminal region of ORF of the candidate target gene at its 5′ terminal. At this time, the primer is designed such that an inducible promoter and a candidate target gene are operably linked. PCR conditions may be appropriately determined in consideration of e.g., the amplification size, base lengths of primers, GC content and Tm value. The resultant PCR amplification product may be isolated and purified, if necessary, in accordance with a routine method. In this manner, a DNA cassette containing homologous region A, a regulatory region containing an inducible promoter, and the like, and homologous region B, can be obtained.

Alternatively, the DNA cassette can be obtained by the following method. Homologous regions A and B are separately amplified by using the genomic DNA of a cell to be used for homologous recombination as a template and appropriate primers, respectively. An amplified DNA fragment of homologous region A is inserted into a site upstream of the regulatory region of an expression vector containing the regulatory region; whereas an amplified DNA fragment of homologous region B is inserted into a site downstream of the inducible promoter of the expression vector containing the regulatory region such that the promoter and the candidate target gene are operably linked, for example, by using a restriction enzyme. In this manner, an expression vector containing a DNA cassette can be obtained.

The lengths of homologous regions A and B are not particularly limited as long as they satisfy the length of bases sufficient to cause homologous recombination. The lengths are each usually about 10 bp or more, preferably about 50 bp or more, more preferably about 100 bp or more, and further preferably about 500 bp or more; and, usually about 10 kbp or less, preferably about 5 kbp or less, and more preferably about 3 kbp or less. In other words, the lengths of homologous regions A and B are each usually from about 10 bp to about 10 kbp, preferably from about 50 bp to about 5 kbp, more preferably from about 100 bp to about 3 kbp, and further preferably, from about 500 ho to about 3 kbp. The lengths of homologous regions A and B may be appropriately set in consideration of the type of cell for use in homologous recombination, and the length of DNA to be inserted.

Subsequently, the DNA cassette or a linear fragment, which is prepared by, for example, cleaving an expression vector containing the DNA cassette with a restriction enzyme, is introduced into a cell to be subjected for homologous recombination by a known method. Examples of a method for introduction to a cell include, but are not limited to, a calcium phosphate method, a lithium acetate method, a lipofection method, a DEAE-dextran method, a protoplast method, an electroporation method, a micro-injection method and a method using a virus vector. The introduction method may be appropriately selected in consideration of the type of cell and introduction efficiency. Thereafter, selection through screening is carried out using a transformation marker to isolate a modified cell in which homologous recombination occurred in the homologous regions A and B of the genome of the cell and an inducible promoter is introduced into a site upstream of the candidate target gene, namely, a modified cell in which the original promoter of a target gene is replaced with the inducible promoter. Whether or not the inducible promoter is integrated into a desired position may be checked by, e.g., a PCR method using genomic DNA as a template. In this manner, a modified cell containing an inducible promoter inserted into a site upstream of the candidate target gene is obtained.

The “cell” to be used in the present invention is not limited as long as it is a cell obtained by suppressing expression of a candidate target gene by RNA interference. More specifically, the cell is preferably a cell in which the expression level of a candidate target gene has been lowered by RNA interference compared to the expression level of the gene in a cell in which expression of the candidate target gene is not regulated and the sensitivity to a test substance has been enhanced compared to that of a cell of the corresponding wild strain.

The RNA interference herein refers to a phenomenon in which introduction of double stranded RNA induces decomposition of mRNA having a complementary sequence to the double stranded RNA to suppress gene expression. The double stranded RNA to be used for RNA interference can be appropriately selected by those skilled in the art depending on the sequence of a target gene and put in use.

The level of biological activity by regulating expression of the candidate target gene in the cell is preferably about 50% or more of the level of biological activity in the cell in which expression of the candidate target gene is not regulated, more preferably about 60% or more, and further preferably about 70% or more; and preferably less than 100%, and more preferably about 98% or less. In other words, the level of biological activity is preferably about 50% or more and less than 100%, more preferably about 60% or more and less than 100%, and further preferably about 70% or more and about 98% or less.

The cell from which the modified cell to be used in the present invention is derived or the cell to be used in the present invention may be either a prokaryotic cell or an eukaryotic cell and appropriately selected depending on the medicinal effect of a test substance. Examples of the prokaryotic cell include a bacterial cell, and an actinomycete cell. Examples of the eukaryotic cell include a fungal cell, an insect cell, an animal cell and a plant cell. For example, if a test substance is an antibacterial drug, a bacterial cell may be selected. If a test substance is an antifungal drug, a fungal cell may be selected. If a test substance is an antineoplastic drug, an animal cell, more specifically, cultured cell of a target malignant tumor may be selected. If a test substance is an antiviral drug, a host cell infected with a virus in which a candidate target gene and/or a cis-element thereof has been modified, may be used. Alternatively, for drug repositioning, a cell different from the cell usually presumed from the medicinal effect of a test substance may be used.

In the present invention, the “biological activity” refers to cellular activity quantitatively determined by experiment. Examples of the cellular activity include cellular metabolic activity, DNA synthetic activity, proliferative activity and respiratory activity. The biological activity can be evaluated by a known method in accordance with the type of cell and the activity to be evaluated. As the biological activity, proliferative activity is preferred, which can be measured by, for example, MTT method, XTT method, WST-1 method, a cell-counting method, a colony method, a turbidity method, a real time PCR method or flow cytometry.

The concentration of a test substance to be used in the present invention is larger than 0 and a concentration not exceeding 10 times of the concentration at which 50% of the biological activity of a cell in which expression of a candidate target gene is not regulated is inhibited, preferably a concentration at which the inhibition of the biological activity does not exceed 50%, more preferably a concentration at which the inhibition of the biological activity does not exceed 40%, further preferably a concentration at which the inhibition of the biological activity does not exceed 30% and further preferably a concentration at which the biological activity is not inhibited. More specifically, the concentration of the test substance is a concentration that is 0.001 times or more of the concentration (IC₅₀) at which the biological activity of a cell in which expression of a candidate target gene is not regulated is inhibited by 50%, preferably 0.01 times or more of IC₅₀, more preferably 0.1 times or more of IC₅₀, and further preferably 0.25 times or more of IC₅₀; and preferably 10 times or less of IC₅₀, more preferably less than or equal to IC₅₀, and further preferably 0.75 times or less of IC₅₀. In other words, the concentration of the test substance is preferably 0.001 times or more and 10 times or less of IC₅₀, more preferably 0.01 times or more and less than or equal to IC₅₀, further preferably 0.1 times or more and less than or equal to IC₅₀, further preferably 0.25 times or more and 0.75 times or less of IC₅₀. If the concentration of a test substance is less than 0.001 times of IC₅₀, the effect of the test substance on a modified cell or a cell as a measurement target is less likely to appear as reduced biological activity. In contrast, if the concentration of a test substance exceeds 10 times of IC₅₀, it becomes difficult to determine whether a decrease in biological activity of a modified cell or a cell as a measurement target is caused by the effect of the test substance on a candidate target gene or by cytotoxicity of the test substance. Accordingly, the effect of the method of the present invention, i.e., successfully identifying a target gene, is not sufficiently exerted.

IC₅₀ of a test substance can be appropriately determined by a known method depending on, e.g., the type of cell and the biological activity to be evaluated. For example, if the biological activity to be evaluated is cellular proliferation activity, cells in which expression of a candidate target gene is not regulated are cultured in the presence of a test substance serially diluted or absence of the test substance, and thereafter, a graph in which the number of cells, turbidity, or the like, after culture is plotted against the concentration of the test substance is prepared. From the graph thus obtained, on the assumption that the number of cells, turbidity, or the like, in the absence of the test substance is regarded as 100%, the concentration of the test substance at which the number of cells, turbidity, or the like, is inhibited by 50%, may be determined as IC₅₀.

When an inducible promoter induces expression of a gene under control in the absence of an inducer or an inductive stimulus and suppresses expression of the gene under control in a dose dependent manner in the presence of the inducer or inductive stimulus, the concentration of the inducer or the strength of the inductive stimulus to be used in the present invention is preferably 0.001 times or more of IC₅₀ at which the biological activity of a modified cell as a measurement target is inhibited by 50%, more preferably 0.01 times or more of IC₅₀, further preferably 0.1 times or more of IC₅₀, and further preferably 0.25 times or more of IC₅₀; and preferably 10 times or less of IC₅₀, more preferably less than or equal to IC₅₀, and further preferably 0.75 times or less of IC₅₀. In other words, the concentration of the inducer or the strength of the inductive stimulus is preferably 0.001 times or more and 10 times or less of IC₅₀, more preferably 0.01 times or more and less than or equal to IC₅₀, further preferably 0.1 times or more and less than or equal to IC₅₀, and further preferably 0.25 times or more and 0.75 times or less of IC₅₀. If the concentration of an inducer or the strength of an inductive stimulus is less than 0.001 times of IC₅₀, the effect of the inducer or inductive stimulus on a modified cell as a measurement target is less likely to appear as reduced biological activity. In contrast, if the concentration of an inducer or the strength of an inductive stimulus exceeds 10 times of IC₅₀, it becomes difficult to determine whether a decrease in biological activity of a modified cell as a measurement target is caused by the effect of the inducer or by inductive stimulus on a candidate target gene or cytotoxicity of the inducer or inductive stimulus. Accordingly, even if an inducer or an inductive stimulus and a test substance are applied to a modified cell, the effects of them do not significantly appear, more specifically, a synergetic effect is not produced, and thus the effect of the method of the present invention, i.e., successfully identifying a target gene, is not sufficiently exerted. Note that, if the biological activity of a modified cell in the absence of an inducer or an inductive stimulus is decreased compared to that of a cell in which expression of a candidate target gene is not regulated, preferably decreased to 50% or less, more preferably decreased to from 50% to 90%, and more preferably decreased to from 50% to 70%, an inducer or an inductive stimulus may not be added or applied.

When an inducible promoter suppresses expression of a gene under control in the absence of an inducer or an inductive stimulus and induces expression of the gene under control in a dose-dependent manner in the presence of the inducer or the inductive stimulus, the concentration of the inducer or the strength of the inductive stimulus is, preferably equal to or more than the concentration or strength (ECs) at which the biological activity of a modified cell as a measurement target is decreased to 50% of a maximum, and preferably 100 times or less of EC₅₀, and more preferably 10 times or less of EC₅₀. In other words, the concentration of the inducer or the strength of the inductive stimulus is preferably EC₅₀ or more and 100 times or less of EC₅₀, and more preferably EC₅₀ or more and 10 times or less of EC₅₀. If the concentration of an inducer or the strength of an inductive stimulus is less than EC₅₀, it becomes difficult to determine whether a decrease in biological activity of a modified cell as a measurement target is caused by the effect of the inducer or inductive stimulus on a candidate target gene or by inhibition of growth of the cell simply by shortage of the inducer or inductive stimulus. In contrast, if the concentration of an inducer or the strength of an inductive stimulus exceeds 100 times of EC₅₀, the effect of the inducer or inductive stimulus on a modified cell as a measurement target is less likely to appear as reduced biological activity. Accordingly, even if an inducer or an inductive stimulus and a test substance are applied to a modified cell, the effects of them do not significantly appear, more specifically, a synergetic effect is not produced, and thus the effect of the method of the present invention, i.e., successfully identifying a target gene, is not sufficiently exerted.

IC₅₀ of an inducer or an inductive stimulus can be appropriately determined by a known method depending on, e.g., the type of cell and the biological activity to be evaluated. For example, if the biological activity to be evaluated is cellular proliferation activity, modified cells as a measurement target are cultured in the presence of an inducer serially diluted or an inductive stimulus serially changed in strength or absence thereof, and thereafter, a graph in which the number of cells, turbidity, or the like, after culture is plotted against the concentration of the inducer or the strength of the inductive stimulus is prepared. From the graph thus obtained, on the assumption that the number of cells, turbidity, or the like, in the absence of the inducer or the inductive stimulus is regarded as 100%, the concentration of the inducer or inductive stimulus at which the number of cells, turbidity, or the like, is inhibited by 50%, may be determined as IC₅₀. Calculation methods of IC₅₀ of a test substance and an inducer or an inductive stimulus may be the same or different. In view of accuracy, the calculation methods are preferably the same. IC₅₀ of an inducer or an inductive stimulus is the concentration determined per type of modified cell, and thus can be different between modified cells having different candidate target genes.

EC₅₀ of an inducer or an inductive stimulus can be appropriately determined by a known method depending on, e.g., the type of cell and the biological activity to be evaluated. For example, if the biological activity to be evaluated is cellular proliferation activity, modified cells as a measurement target are cultured in the presence of an inducer serially diluted or an inductive stimulus serially changed in strength or absence thereof, and thereafter, a graph in which the number of cells, turbidity, or the like, after culture is plotted against the concentration of the inducer or the strength of the inductive stimulus is prepared. From the graph thus obtained, the concentration or strength at which a half of a maximum reaction is shown may be determined as EC₅₀. EC₅₀ of an inducer or an inductive stimulus is a concentration determined per type of modified cell, and thus can be different between modified cells having different candidate target genes.

To identify a target gene of a test substance by using the method of the present invention, a modified cell in which at least one selected from the group consisting of a candidate target gene, a cis-element thereof and a trans-element thereof has been modified or a cell in which expression of a candidate target gene has been suppressed by RNA interference is cultured in the presence of a test substance of the predetermined concentration as mentioned above and a modified cell or a cell having a decreased biological activity may be detected.

A method for culturing a modified cell or a cell (hereinafter referred to as a modified cell or the like) is not particularly limited, and a modified cell or the like may be inoculated to a culture medium, which varies depending on the type of cell and the biological activity measured, and cultured in the presence of a test substance of a predetermined concentration in accordance with a routine method. A test substance may be added to a culture medium by directly adding it to a liquid medium, adding it in a solid medium, smearing it onto a solid medium, or the like. The addition timing may be before or after inoculation of a modified cell or the like. The culture time may be appropriately set depending on e.g., the type of cell and the biological activity to be evaluated, and usually from about 3 hours to about 7 days, preferably from about 12 hours to about 2 days, and further preferably from about 12 to about 24 hours.

After culturing a modified cell or the like, biological activity of the modified cell or the like is measured. If the biological activity is low compared to the case where a modified cell or the like is cultured in the absence of a test substance or the case where a cell in which expression of a candidate target gene is not regulated is cultured in the presence of a test substance of the same concentration, the candidate target gene in the modified cell or the like can be determined as the target gene of a test substance.

Alternatively, when biological activity of a modified cell or the like is measured after culturing the modified cell or the like, if the biological activity is the lowest in those of other modified cells or the like having different candidate target genes and cultured in the presence of a test substance of a predetermined concentration, a candidate target gene of the modified cell can be determined as the target gene of the test substance. Note that, if a molecule encoded by a candidate target gene of a modified cell having the lowest biological activity and a molecule encoded by a candidate target gene of a modified cell (at least one type) having the second lowest biological activity are mutually associated, one of them activates or suppresses the other, one of them is required for constructing a molecule activating or suppressing the other, or the like, then these candidate target genes can be determined as the target genes. For example, when overexpression strains thereof are prepared and if a strain exhibits low sensitivity to a test substance due to overexpression, the candidate target gene of the strain can be determined as the target gene whereas if a strain exhibits no change in sensitivity, the candidate target gene of the strain can be determined as a target related gene.

If a modified cell is prepared by inserting an inducible promoter into a site upstream of the candidate target gene, to identify a target gene of a test substance, the modified cell is cultured in the presence of an inducer of the predetermined concentration mentioned above or an inductive stimulus of the predetermined strength mentioned above and a test substance of the predetermined concentration mentioned above, and then, a modified cell whose biological activity decreases may be detected.

The method for culturing a modified cell is not particularly limit. A modified cell may be inoculated in the culture medium, which varies depending on the type of cell and the biological activity measured, and cultured in the presence of an inducer of a predetermined concentration or an inductive stimulus of a predetermined strength and a test substance of a predetermined concentration in accordance with a routine method. An inducer and a test substance may be added to a culture medium by directly adding them to a liquid medium, adding them in a solid medium, smearing them onto a solid medium, or the like. The addition timing may be before or after inoculation of a modified cell. The order of addition is not limited. The inductive stimulus may be applied to the culture medium containing a modified cell depending on the type of stimulus. The culture time may be appropriately set depending on, e.g., the type of cell and the biological activity to be evaluated, and is usually from about 3 hours to about 7 days, preferably from about 12 hours to about 2 days, and further preferably from about 12 to about 24 hours.

After culturing a modified cell, the biological activity of the modified cell is measured. If the biological activity is low compared to the case where the modified cell is cultured in the presence of an inducer of the same concentration alone or an inductive stimulus of the same strength alone and/or the case where the modified cell is cultured in the presence of a test substance of the same concentration alone, the candidate target gene in the modified cell can be determined as the target gene of the test substance.

Alternatively, after culturing a modified cell, the biological activity of the modified cell is measured. If the biological activity is the lowest in those of other modified cells having different candidate target genes and cultured in the presence of an inducer of a predetermined concentration or an inductive stimulus of a predetermined strength and a test substance of a predetermined concentration, the candidate target gene of the modified cell can be determined as the target gene of the test substance. Note that, if a molecule encoded by a candidate target gene of a modified cell having the lowest biological activity and a molecule encoded by a candidate target gene of a modified cell (at least one type) having the second lowest biological activity are mutually associated, one of them activates or suppresses the other, one of them is required for constructing a molecule activating or suppressing the other, or the like, then these candidate target genes can be determined as the target genes. For example, when overexpression strains thereof are prepared and if a strain exhibits low sensitivity to a test substance due to overexpression, the candidate target gene of the strain can be determined as the target gene whereas if a strain exhibits no change in sensitivity, the candidate target gene of the strain can be determined as a target related gene.

If a modified cell is prepared by inserting an inducible promoter into a site upstream of a candidate target gene and the biological activity of the modified cell decreases to 50% or less of the biological activity of a cell in which expression of the candidate target gene is not regulated in the absence of an inducer or an inductive stimulus, in order to identify a target gene of a test substance, the modified cell is cultured in the presence of the test substance of the predetermined concentration mentioned above, and then, a modified cell whose biological activity decreases may be detected.

A method for culturing a modified cell is not particularly limited. A modified cell is inoculated to a culture medium, which varies depending on the type of cell and the biological activity measured, and cultured in the presence of a test substance of a predetermined concentration in accordance with a routine method. A test substance may be added to a culture medium by directly adding it to a liquid medium, adding it in a solid medium, smearing it onto a solid medium, or the like. The addition timing may be before or after inoculation of a modified cell. The culture time may be appropriately set depending on, e.g., the type of cell and the biological activity to be evaluated, and is usually about 3 hours to about 7 days, preferably about 12 hours to about 2 days and further preferably about 12 to about 24 hours.

After culturing a modified cell, the biological activity of the modified cell is measured. If the biological activity is low compared to the case where the modified cell is cultured in the absence of a test substance or the case where a cell in which expression of a candidate target gene is not regulated is cultured in the presence of the test substance of the same concentration, the candidate target gene of the modified cell can be determined as the target gene of the test substance.

Alternatively, after culturing a modified cell, the biological activity of the modified cell is measured. If the biological activity is the lowest in those of other modified cells having different candidate target genes and cultured in the presence of a test substance of a predetermined concentration, the candidate target gene of the modified cell can be determined as the target gene of the test substance. Note that, if a molecule encoded by a candidate target gene of a modified cell having the lowest biological activity and a molecule encoded by a candidate target gene of a modified cell (at least one type) having the second lowest biological activity are mutually associated, one of them activates or suppresses the other, one of them is required for constructing a molecule activating or suppressing the other, or the like, then these candidate target genes can be determined as the target genes. For example, when overexpression strains thereof are prepared and if a strain exhibits low sensitivity to a test substance due to overexpression, the candidate target gene of the strain can be determined as the target gene whereas if a strain exhibits no change in sensitivity, the candidate target gene of the strain can be determined as a target related gene.

In an embodiment of the present invention, a modified cell or the like is cultured in the presence of an inducer of a predetermined concentration or an inductive stimulus of a predetermined strength, in the presence of a test substance of a predetermined concentration, and in the presence of an inducer of a predetermined concentration or an inductive stimulus of a predetermined strength and a test substance of a predetermined concentration, and then, the biological activity of each of them is measured to calculate a synergy index. The synergy index may be calculated by use of a formula for computation known per se. As the formula for computation, the following formula is mentioned; however, the formula is not limited to this.

Synergy index=(A+B)/2C (1)

(where A represents biological activity when a cell is cultured in the presence of an inducer or an inductive stimulus; B represents the biological activity when a cell is cultured in the presence of a test substance; and C represents the biological activity when a cell is cultured in the co-presence of an inducer or an inductive stimulus and a test substance.)

The value of the biological activity herein varies depending on the evaluation item. For example, if cell proliferation is used as index, a measured value or a deviation value of the measured value of e.g., the number of cells and turbidity can be used.

Modified cells or the like having different genes as the candidate target genes are also subjected to the above operation. The resultant synergy indexes are compared to detect a modified cell or the like having the highest synergy index. The candidate target gene of the modified cell or the like thus detected can be determined as the target gene of a test substance. Note that, if the molecules encoded by the candidate target gene of the modified cell or the like showing the highest synergy index and the modified cell or the like (at least one) showing the second highest synergy index are mutually associated, one of them activates or suppresses the other, one of them is required for constructing a molecule activating or suppressing the other, or the like, then these candidate target genes can be determined as the target genes altogether.

The above embodiment can be carried out by inoculating modified cells or the like having different genes as the candidate target genes to wells of, for example, a 96-well plate, culturing in the presence of an inducer of a predetermined concentration or an inductive stimulus of a predetermined strength, a test substance of a predetermined concentration, or an inducer of a predetermined concentration or an inductive stimulus of a predetermined strength and a test substance of a predetermined concentration for a predetermined period, measuring the biological activity of each well after culture, calculating a synergy index per type of modified cell and detecting a modified cell or the like having the highest synergy index from the calculation results. As modified cells or the like having different genes as the candidate target genes, desired modified cells or the like may be selected and put in use or a library per species may be prepared and put in use. Examples of the library include a library containing modified cells or the like with respect to all genes of a target species, a library containing modified cells or the like with respect to genes influencing growth of a target species and a library containing modified cells or the like with respect to genes essential for growth of a target species.

In the present invention, when the concentration of a test substance, more preferably the concentration of an inducer or the strength of an inductive stimulus and the concentration of a test substance are separately specified to fall within predetermined ranges per modified cell or the like, the effect of the test substance on the modified cell or the like having a target gene as a candidate target gene is produced, more preferably, the effect of an inducer or an inductive stimulus and the effect of a test substance are synergistically produced, with the result that the biological activity of the modified cell or the like significantly decreases. By detecting the decrease, the target gene of a test substance can be efficiently identified.

In Examples described later, as a suitable embodiment of the method according to the present invention, a method for identifying the target gene of a test substance by using a recombinant fungal cell prepared by inserting an inducible promoter into a site upstream of the candidate target gene is described. More specifically, a method for identifying the target gene of an antifungal drug by using a tetracycline transcriptional repression strain (hereinafter may be referred to also as Tet strain) cell of Candida glabrata, is described.

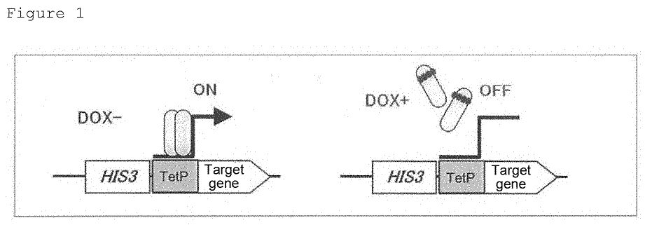

In Tet-Off (registered trademark) system (FIG. 1), expression of a gene of interest is regulated by the presence or absence of tetracycline or a derivative thereof. In this system, a tetracycline-regulated transactivator (rTA) containing a Tet repressor (TetR) binds to a Tet operator (TetO) to activate a promoter in the absence of tetracycline or a derivative thereof to induce transcription of a gene under control of the promoter; whereas, in the presence of tetracycline or a derivative thereof, since binding of rTA to TetO is inhibited by tetracycline or a derivative thereof, transcriptional activation by the promoter does not occur, with the result that transcription of the gene under control of the promoter is suppressed. As described, in the Tet-Off system, transcription of a desired gene can be regulated just by adding tetracycline or a derivative thereof externally. Tetracycline or a derivative thereof, since it is less toxic to fungi and host animals thereof, is suitable for regulating expression of genes of eukaryotic cells including fungi. In addition, since expression of a gene of interest can be completely suppressed in a short time, the system is useful.

(1) Preparation of Tet Strain

Candida glabrata Tet strain can be constructed, for example, as follows.

First, an expression vector, in which rTA containing TetR and a transcription factor is operably linked to a site downstream of a constitutive promoter, is prepared. The constitutive promoter refers to a promoter which expresses a gene under control thereof irrelevant to growth conditions of a host cell. Examples of the constitutive promoter include a promoter derived from Candida glabrata and a promoter derived from Saccharomyces cerevisiae, which is a genealogically related species to Candida glabrata. The expression vector is introduced into Candida glabrata in accordance with a routine method, and thereafter, a cell having the expression vector introduced therein is selected to obtain a Candida glabrata strain expressing rTA.

Separately, an expression vector containing a chimeric promoter (Tet promoter), in which TetO and a minimal promoter are linked, and a transformation marker upstream of the chimeric promoter, is prepared. As the minimal promoter, a promoter of a gene whose expression is always suppressed except the meiosis period such that the expression suppression state can be maintained in the presence of doxycycline, is preferably used. Then, PCR is carried out using the expression vector as a template and a primer pair amplifying a region containing the transformation marker gene and the chimeric promoter, more specifically a pair of a primer, in which a DNA sequence homologous to genomic DNA of the 5′ flanking region of a candidate target gene is added to the 5′ terminal, and a primer in which a DNA sequence homologous to genomic DNA of the 5′ terminal region of ORF of the candidate target gene is added to the 5′ terminal. At this time, the primer is designed in such a manner that the chimeric promoter and the candidate target gene are operably linked. In this manner, a DNA cassette containing the homologous region, transformation marker gene, chimeric promoter and homologous region can be obtained.

The DNA cassette obtained is introduced into the Candida glabrata strain expressing rTA and integrated in a host genome by homologous recombination in accordance with a routine method, and then, a cell (Tet strain) where homologous recombination occurred is selected by using the transformation marker. Whether the chimeric promoter is integrated to a desired site of the genome can be checked by PCR using genomic DNA as a template.

(2) Determination of Concentrations of Doxycycline and Test Substance

The Tet strain obtained in section (1) is cultured in the presence of serially diluted doxycycline or absence thereof. Thereafter, turbidity is measured at 600 nm by a spectrophotometer. Then, a graph in which the turbidity is plotted against the doxycycline concentration is prepared. From the graph, the concentration of doxycycline at which the turbidity is inhibited by 50% is calculated as IC₅₀, on the assumption that the turbidity in the case where the Tet strain is cultured in the absence of doxycycline is regarded as 100%. Note that, if the turbidity in the case where the Tet strain is cultured in the absence of doxycycline is 50% or less compared to the turbidity in the case where Candida glabrata cell, in which expression of a candidate target gene is not regulated, is cultured, the concentration of doxycycline to be used in the method of the present invention is set at 0.

The Candida glabrata cell, in which expression of a candidate target gene is not regulated, is cultured in the presence of a test substance, i.e., an antifungal drug, serially diluted or absence thereof. Thereafter, turbidity is measured at 600 nm by a spectrophotometer. Then, a graph in which the turbidity is plotted against the antifungal-drug concentration is prepared. From the graph, the concentration of the antifungal drug at which the turbidity is inhibited by 50% is calculated as IC₅₀, on the assumption that the turbidity in the case where the cell is cultured in the absence of antifungal drug is regarded as 100%.

A Tet strain and a Candida glabrata cell in which expression of a candidate target gene is not regulated may be inoculated in a culture medium usually used for culturing Candida glabrata and containing assimilable carbon source, nitrogen source and other essential nutrients and subjected to shaking culture or stirred culture under aeration in accordance with a routine method. Examples of the culture medium include SD medium, PDA medium and YPD medium. The pH of the culture medium is preferably adjusted to from about 5 to about 8. The culture temperature is usually from about 20° C. to about 35° C. and preferably from about 25° C. to about 30° C. The culture time is usually from about 10 hours to about 10 days, preferably from about 12 hours to about 5 days and further preferably from about 12 hours to about 2 days.

(3) Culture of Tet Strain

In the presence of doxycycline of a predetermined concentration and the antifungal drug of a predetermined concentration based on IC₅₀ determined in section (2), the Tet strain obtained in section (1) is cultured and the turbidity at CD 600 or OD 660 is measured. The culture conditions of the Tet strain may be substantially the same as those in the step (2).

(4) Identification of Target Gene

With respect to the Tet strain, using turbidity at the time of culture in the presence of doxycycline and the antifungal drug, turbidity at the time of culture in the presence of doxycycline of the same concentration, and turbidity at the time of culture in the presence of the antifungal drug of the same concentration, a synergy index is calculated in accordance with the following formula (2).

Synergy index=(A+B)/2C (2)

(where A represents the turbidity in the case of culture performed in the presence of doxycycline; B represents the turbidity in the case of culture performed in the presence of an antifungal drug; and C represents the turbidity in the case of culture performed in the co-presence of doxycycline and an antifungal drug.)

Tet strains containing genes of Candida glabrata, preferably Tet strains containing genes essential for growth are subjected to the operation mentioned above. The synergy indexes obtained are compared to detect the Tet strain showing the highest synergy index. The candidate target gene in the Tet strain can be determined as the target gene of the antifungal drug. Alternatively, if the molecules encoded by the candidate target gene of the Tet strain showing the highest synergy index and the Tet strain (at least one) showing the second highest synergy index are mutually associated, one of them activates or suppresses the other, one of them is required for constructing a molecule activating or suppressing the other, or the like, then these candidate target genes can be determined as the target genes.

In Examples described later, Candida glabrata is used as a fungus because Candida glabrata is relatively small in genomic size among pathogenic fungi; easily manipulated by genetic engineering; and suitable for genome-wide functional analysis. However, the fungus as an evaluation target is not limited to Candida glabrata. Examples of the fungus include Chytridiomycota; Zygomycetes such as genus Rhizopus and genus Mucor; Ascomycota; Basidiomycota such as genus Cryptococcus (for example, Cryptococcus neoformans), genus Malassezia (for example, Malassezia furfur) and rust fungus; imperfect fungi such as Trichophyton (for example, Trichophyton rubrum, Trichophyton mentagrophytes), genus Sporothrix and Dematiaceae; and yeasts. As Ascomycota, it is understood that the followings can be used; yeasts including budding yeasts such as Trichophyton (for example, Trichophyton rubrum, Trichophyton mentagrophytes), Sporothrix (for example, Sporothrix schenkii), Aspergillus (for example, Aspergilius fumigatus), Pneumocystis (for example, Pneumocytsis jirovecii), Candida (for example, Candida albicans, Candida grabrata) and Saccharomyces (for example, Saccharomyces cerevisiae) and fission yeasts such as genus Schizosaccharomyces; molds such as Peniciliium italicum, Aspergillus oryzae and Neurospora crassa; mushrooms such as morel and truffle, genus Eutypa; Pyricularia oryzae, Blumeria graminis, scab pathogen and rust fungus. Of them, Trichophyton, Sporothrix, Aspergillus, Pneumocystis, Candida, Saccharomyces, and the like, which are known to be pathogenic to humans, are preferably used as a target.

### EXAMPLES

Now, the present invention will be more specifically described by way of Examples; however, the present invention is not limited to these.

### Example 1 Identification of Target Gene of Antifungal Drug, Fluconazole, Using Candida glabrata Tet Strain (1)

(1) Preparation of Tet Strain

Tetracycline transcriptional repression strains (Tet strains), were each prepared by inserting Tet-Off promoter into a site upstream of each of the genes of Candida glabrata, in accordance with the method described in Ueno K, Uno J, Nakayama H, Sasamoto K, Mikami Y, Chibana H. Development of a highly efficient gene targeting system induced by transient repression of YKU80 expression in Candida glabrata. Eukaryot Cell. 2007; 6: 1239-1247.

(2) Determination of 50% Inhibitory Concentrations of Doxycycline and Fluconazole

SD medium (6.7 g/L yeast nitrogen base, 2% glucose) (100 μL) was added dropwise to a 96-well cell culture plate. To the plate, doxycycline (Dox) serially diluted was added, and then, the Tet strain prepared in the step (1) was inoculated. The strain was cultured at 30° C. for 20 hours and turbidity was measured at CD 600. The concentration at which the turbidity of the Tet strain decreased, by addition of Dox, to about 50% of that in the case where culture was carried out in the absence of Dox was specified as IC₅₀. Herein, for example, IC₅₀ or the Tet strain having ERG11 gene as a candidate target gene for Dox was 1 μM.

Separately, SD medium (6.7 g/L yeast nitrogen base, 2% glucose) (100 μL) was added dropwise to a 96-well cell culture plate. To the plate, fluconazole (Flu) serially diluted was added, and a cell strain (wild strain: CBS138 strain) in which expression of a candidate target gene is not regulated was inoculated. The cell strain was cultured at 30° C. for 20 hours and turbidity was measured at OD 600. The concentration at which the turbidity of CBS138 strain decreased, by addition of Flu, to about 50% of that in the case where culture was carried out in the absence of Flu was specified as IC₅₀. The IC₅₀ of CBS138 strain for Flu was 50 μM.

(3) Culture of Tet Strain

SD medium (6.7 g/L yeast nitrogen base, 2% glucose) (100 μL), to which Dox in a concentration of 3/10 times of IC₅₀, which was calculated in the step (2) with respect to each Tet strain, and/or Flu (25 μM) were added, was added dropwise to a 96 well cell culture plate. For example, if Tet strain having ERG11 gene as a candidate target gene was used, the concentration of Dox was set at 0.3 μM. To the wells of the plate, the Tet strains prepared in the step (1) were inoculated. The Tet strains were cultured at 30° C. for 20 hours and turbidity was measured at OD 600.

(4) Calculation of Synergy Index

The synergy index of each of the Tet strains was calculated by using the turbidity of the culture carried out in the presence of Dox and 25 μM Flu, the turbidity of the culture carried out in the presence of Dox of the same concentration and the turbidity of the culture carried out in the presence of Flu of the same concentration in accordance with the formula (3) below.

Synergy index=(A+B)/2C (3)

(where A represents the turbidity of culture carried out in the presence of Dox; B represents the turbidity of culture carried out in the presence of Flu; and C represents the turbidity of culture carried out in the co-presence of Dox and Flu.)

(5) Results

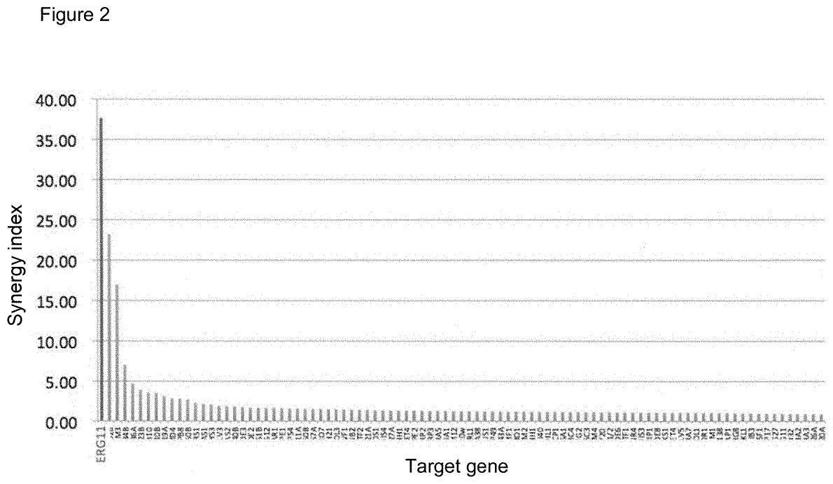

The synergy indexes calculated in the step (4) are shown in FIG. 2. From the results, it was found that the synergy index of Tet strain having ERG11 gene as a candidate target gene was significantly high compared to those of other Tet strains. From this, it was considered that ERG11 gene is the target gene of fluconazole. Actually, it is known that ERG11 gene is the target gene of fluconazole. Thus, it was confirmed that the target gene of a test substance, i.e., an antifungal drug, fluconazole, can be identified by the method of the present invention.

### Example 2 Identification of Target Gene of Antifungal Drug, Fluconazole, Using Candida glabrata Tet Strain (2)

The Tet strains prepared in Example 1 (1) were each cultured in substantially the same manner as in the method of Example 1 (3) except that Dox of a concentration of 3/100 times of IC₅₀ calculated in Example 1 (2) and/or 7.5 μM Flu were used. At this time, for example, if Tet strain having ERG11 gene as a candidate target gene was used, the concentration of Dox was set at 0.03 μM. Thereafter, the synergy indexes of the Tet strains were calculated in accordance with the method of Example 1 (4).

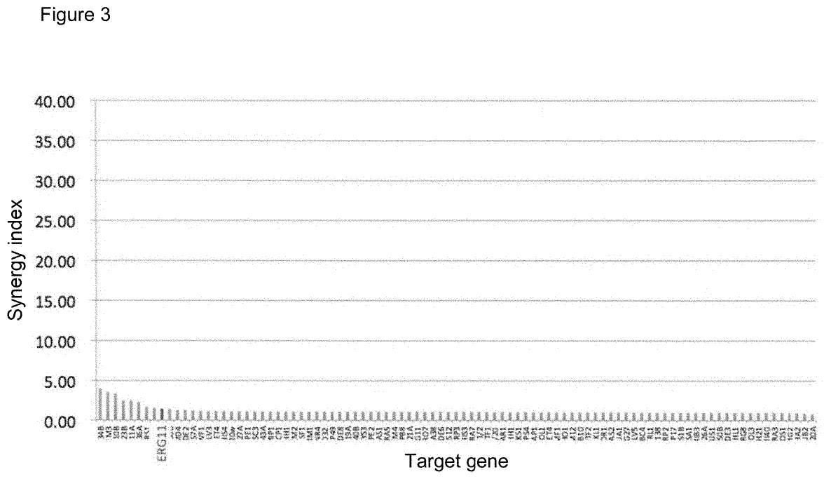

The synergy indexes calculated are shown in FIG. 3. From the results, it was confirmed that ERG11 gene known as the target gene of fluconazole shows a high synergy index.

### Example 3 Identification of Target Gene of Antifungal Drug, Fluconazole, Using Candida glabrata Tet Strain (3)

The Tet strains prepared in Example 1 (1) were each cultured in substantially the same manner as in the method of Example 1 (3) except that Dox of a concentration of 3/1000 times of IC₅₀ calculated in Example 1 (2) and/or 25 μM Flu were used. At this time, for example, if Tet strain having ERG11 gene as a candidate target gene was used, the concentration of Dox was set at 0.003 μM. Thereafter, the synergy indexes of the Tet strains were calculated in accordance with the method of Example 1 (4).

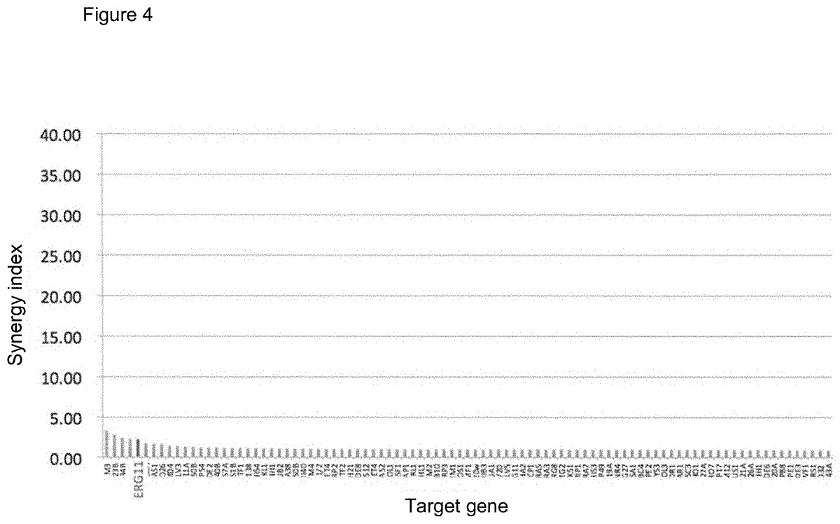

The synergy indexes calculated are shown in FIG. 4. From the results, it was confirmed that ERG11 gene known as the target gene of fluconazole shows a high synergy index.

### Example 4 Identification of Target Gene of Antifungal Drug, Fluconazole, Using Candida glabrata Tet Strain (4)

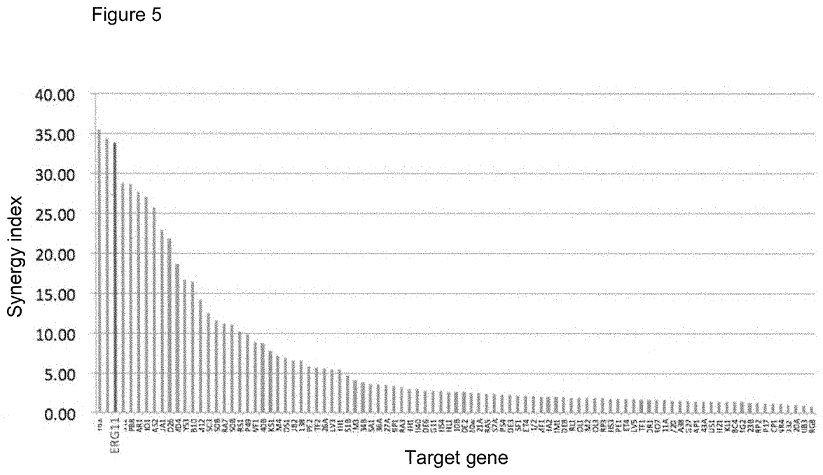

The Tet strains prepared in Example 1 (1) were cultured in substantially the same manner as in the method of Example 1 (3) except that Dox of a concentration of IC₅₀ calculated in Example 1 (2) and/or 50 μM Flu were used. At this time, for example, if Tet strain having ERG11 gene as a candidate target gene was used, the concentration of Dox was set at 1 μM. Thereafter, the synergy indexes of the Tet strains were calculated in accordance with the method of Example 1 (4) The synergy indexes calculated are shown in FIG. 5.

From the results, it was confirmed that ERG11 gene known as the target gene of fluconazole shows a high synergy index.

### Example 5 Identification of Target Gene of Antifungal Drug, Terbinafine, Using Candida glabrata Tet Strain

(1) Determination of 50% Inhibitory Concentrations of Doxycycline and Terbinafine

SD medium (6.7 g/L yeast nitrogen base, 2% glucose) (100 μL) was added dropwise to a 96-well cell culture plate. To the plate, doxycycline (Dox) serially diluted was added and each of the Tet strains prepared in Example 1 (1) was inoculated. The strains were each cultured at 30° C. for 20 hours and turbidity was measured at OD 600. The concentration at which the turbidity of the Tet strain decreased, by addition of Dox, to about 50% of that in the case where culture was carried out in the absence of Dox was specified as IC₅₀. Herein, for example, IC₅₀ of the Tet strain having ERG1 gene as a candidate target gene for Dox was 0.5 LM.

Separately, SD medium (6.7 g/L yeast nitrogen base, 2% glucose) (100 μL) was added dropwise to a 96-well cell culture Plate. To the plate, terbinafine (Ter) serially diluted was added, and a cell strain (wild strain: CBS138 strain) in which expression of a candidate target gene is not regulated was inoculated. The cell strain was cultured at 30° C. for 20 hours and turbidity was measured at OD 600. The concentration at which the turbidity of CBS138 strain decreased, by addition of Ter, to about 50% of that in the case where culture was carried out in the absence of Ter was specified as IC₅₀. The IC₅₀ of CBS138 strain for Ter was 48 μM.

(2) Culture of Tet Strain

The Tet strains prepared in Example 1 (1) were each cultured in substantially the same manner as in the method of Example 1 (3) except that Dox of a concentration of ½ times of IC₅₀ calculated in Example 5 (1) and/or 12 μM Ter were used. At this time, for example, if Tet strain having ERG1 gene as a candidate target gene was used, the concentration of Dox was set at 0.25 μM.

(3) Calculation of Synergy Index

The synergy indexes were calculated in substantially the same manner as in the method of Example 1 (4) except that the turbidity of the culture carried out in the presence of Dox and 12 μM Ter, the turbidity of the culture carried out in the presence of Dox of the same concentration, and the turbidity of the culture carried out in the presence of Ter of the same concentration for each Tet strain were used.

(4) Results

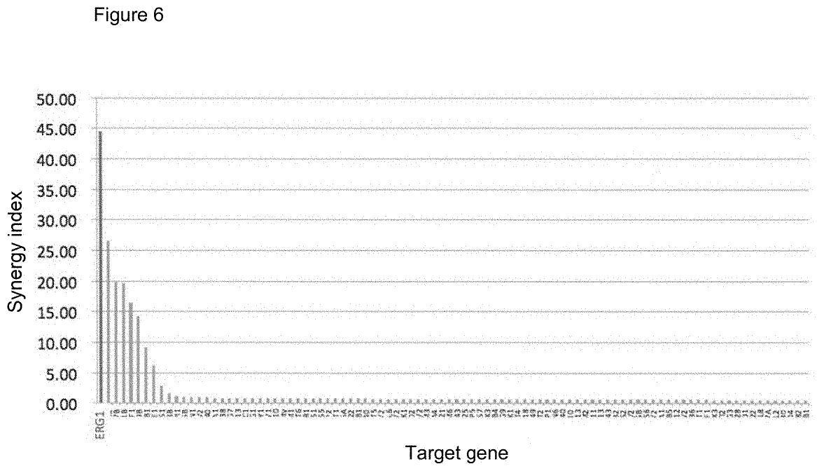

The synergy indexes calculated in the step (3) are shown in FIG. 6. From the results, it was found that the synergy index of Tet strain having ERG1 gene as a candidate target gene is significantly high compared to those of other Tet strains. From this, it was considered that ERG1 gene is the target gene of terbinafine. Actually, it is known that ERG1 gene is the target gene of terbinafine. Thus, it was confirmed that the target gene of a test substance, i.e., an antifungal drug, terbinafine, can be identified by the method of the present invention.

1: A method for identifying a target gene of a test substance previously confirmed to have a predetermined medicinal effect, the method comprising:
culturing a modified cell in which at least one selected from the group consisting of a candidate target gene, a cis-element thereof and a trans-element thereof is modified, or a cell in which expression of a candidate target gene is suppressed by RNA interference, in the presence of the test substance; and
detecting the modified cell or the cell having a decreased biological activity.

2: The method according to claim 1, wherein the modified cell comprises an inducible promoter inserted into a site upstream of the candidate target gene.

3: The method according to claim 2, wherein the culturing of the modified cell comprises culturing the modified cell in the presence of an inducer in a concentration that is from 0.001 times to 10 times of IC₅₀, which is a concentration at which biological activity of the modified cell is inhibited by 50%, and the test substance in a concentration that is from 0.001 times to 10 times of IC₅₀, which is a concentration at which biological activity of a cell in which expression of the candidate target gene is not regulated is inhibited by 50%.

4: The method according to claim 2, wherein the culturing of the modified cell comprises culturing the modified cell in the presence of the test substance in a concentration that is from 0.001 to 10 times of IC₅₀, which is a concentration at which biological activity of a cell in which expression of the candidate target gene is not regulated is inhibited by 50% when biological activity of the modified cell decreases to 50% or less compared to the cell in which expression of the candidate target gene is not regulated.

5: The method according to claim 2, wherein the culturing of the modified cell comprises culturing the modified cell in the presence of an inducer in a concentration that is not less than EC₅₀, which is a concentration at which biological activity of the modified cell decreases to 50% of a maximum, and 100 times or less of EC₅₀ and the test substance in a concentration that is from 0.001 times to 10 times of IC₅₀, which is a concentration at which biological activity of a cell in which expression of the candidate target gene is not regulated is inhibited by 50%.

6: The method according to claim 3, wherein the concentration of the inducer is 0.01 times or more of IC₅₀ and less than or equal to IC₅₀.

7: The method according to claim 3, wherein the inducible promoter is a Tet-Off promoter.

8: The method according to claim 1, wherein the test substance has at least one medicinal effect of at least one drug selected from the group consisting of an antibacterial drug, an antifungal drug, an antineoplastic drug and an antiviral drug.

9: The method according to claim 1, wherein the decreased biological activity is a decreased cell proliferative activity.

10: The method according to claim 1, wherein the cell is an eukaryotic cell.

11: The method according to claim 1, wherein the cell is a fungal cell.

12: The method according to claim 4, wherein the inducible promoter is a Tet-Off promoter.

13: The method according to claim 2, wherein the test substance has at least one medicinal effect of at least one drug selected from the group consisting of an antibacterial drug, an antifungal drug, an antineoplastic drug and an antiviral drug.

14: The method according to claim 3, wherein the test substance has at least one medicinal effect of at least one drug selected from the group consisting of an antibacterial drug, an antifungal drug, an antineoplastic drug and an antiviral drug.

15: The method according to claim 2, wherein the decreased biological activity is a decreased cell proliferative activity.

16: The method according to claim 3, wherein the decreased biological activity is a decreased cell proliferative activity.

17: The method according to claim 2, wherein the cell is an eukaryotic cell.

18: The method according to claim 3, wherein the cell is an eukaryotic cell.

19: The method according to claim 2, wherein the cell is a fungal cell.

20: The method according to claim 3, wherein the cell is a fungal cell.
